(19) United States
(12) Patent Application Publication
(10) Pub. No.: US 2010/0068771 A1
(43) Pub. Date: Mar. 18, 2010

(54) PROCESS FOR THE PREPARATION OF ENANTIOMERICALLY ENRICHED BETA-AMINO ALCOLHOLS STARTING FROM GLYCINE AND AN ALDEHYDE IN THE PRESENCE OF A THREONINE ALDOLASE AND A DECARBOXYLASE

(76) Inventors: Martin Schürmann, Jülich (DE); Daniel Mink, Eupen (DE); Michael Wolberg, Neutraubling (DE)

Correspondence Address: NIXON & VANDERHYE, PC, 901 NORTH GLEBE ROAD, 11TH FLOOR, ARLINGTON, VA, 22203, US

(21) Appl. No.: 12/516,953

(22) PCT Filed: Apr. 12, 2007
(86) PCT No.: PCT/EP07/03274

§ 371 Date: Nov. 12, 2009

(30) Foreign Application Priority Data
Apr. 13, 2006 (EP) 06007875.5

(51) Int. Cl. C12P 13/00

(52) U.S. Cl. 435/128

(57) ABSTRACT

The invention relates to a process for the preparation of an enantiomerically enriched β-amino alcohol, wherein glycine or a glycine salt and an aldehyde are reacted in the presence of a threonine aldolase and a decarboxylase to form the corresponding enantiomerically enriched β-aminoalcohol, and wherein at least either the threonine aldolase or the decarboxylase is β-selective. In a preferred embodiment of the invention at least either the threonine aldolase or the decarboxylase is enantioselective.

The invention relates to a process for the enzymatic preparation of an enantiomerically enriched β-aminoalcohol. Enantiomerically enriched β-aminoalcohols are important pharmaceuticals or precursors thereof, e.g. for the treatment of cardiovascular diseases, cardiac failure, asthma, and glaucoma. Furthermore, enantiomerically enriched β-aminoalcohols can be used as building blocks for catalysts and chiral resolution agents used in asymmetric synthesis.

Such a process is known from EP-B1-0 751 224, wherein a process is disclosed for the preparation of (R)-2-amino-1-phenylethanol or its halogen substitution products by conversion of DL-threo-3-phenylserine or its halogen substitution products with an L-selective tyrosine decarboxylase, which tyrosine decarboxylase is preferably derived from Enterococcus, Lactobacillus, Providencia, Fusarium or Gibberella.

A major disadvantage of this process is that in converting a racemic starting material using an enantioselective enzyme a maximum yield of 50% of the enantiomerically pure endproduct can be reached.

Therefore, it is the object of the invention to provide a process in which the maximum may be higher.

This object is achieved by a process, wherein glycine or a glycine salt and an aldehyde are reacted in the presence of a threonine aldolase and a decarboxylase to form the corresponding enantiomerically enriched β-aminoalcohol, wherein at least either the threonine aldolase or the decarboxylase is β-selective.

As is shown in the examples, with the process of the invention, the β-aminoalcohol can be prepared with a high enantiomeric excess (e.e.) in a yield higher than 50%.

An enzymatic process for the preparation of β-hydroxy-α-amino acids by reacting glycine with a wide range of aldehydes in the presence of an enantioselective threonine aldolase is known from Kimura et al (1997), J. Am. Chem. Soc. Vol 199, pp 11734-11742. This enzymatic process has the disadvantage that the preference of the enzyme to prepare either the threo or the erythro form of the (β-hydroxy-α-amino acid, is markedly low. Another drawback of this process is that generally, low yields are obtained.

It is surprising that the process of the present invention can lead to high yields of the enantiomerically enriched β-aminoalcohol, since as is disclosed by Kimura et al (1997), J. Am. Chem. Soc. Vol 199, pp 11734-11742 low yields of β-hydroxy-α-amino acid are obtained by reacting glycine and an aldehyde in the presence of an enantioselective threonine aldolase. Furthermore, as indicated above, in a process according to EP-B1-0 751 224, the maximal yield of β-amino alcohol by decarboxylation of β-hydroxy-α-amino acid using an enantioselective tyrosine decarboxylase is only 50%. It is therefore surprising that by combining these two processes, the overall yield is higher than when these two processes would be performed independently of one another.

It is also surprising that with the process of the invention, β-amino alcohols can be prepared with a high e.e. As indicated above, the ratio of the threo:erythro product produced in a process for the preparation of β-hydroxy-α-amino acids by reacting glycine with a wide range of aldehydes in the presence of an enantioselective threonine aldolase is close to one (Kimura et al (1997), J. Am. Chem. Soc. Vol 199, pp 11734-11742). A non-β-selective decarboxylation of the formed threo β-hydroxy-α-amino acid respectively of the formed erythro β-hydroxy-α-amino acid would therefore theoretically lead to a mixture of enantiomers of the corresponding β-amino alcohol; the ratios of the enantiomers being also close to one. In other words, one would expect that by combining the enzymatic preparation of β-hydroxy-α-amino acid according to the process of Kimura et al (1997) with decarboxylation of the β-hydroxy-α-amino acid to form the corresponding β-amino alcohol one would obtain a β-amino alcohol with no to low enantiomeric excess. However as is shown in the examples, with the process of the present invention β-amino alcohols may be prepared with a high e.e.

Furthermore, with the process of the present invention, yield of more than 50% can be obtained.

Additional advantages of the process of the invention are for example that the starting materials are often easily accessible and commercially attractive, no chemical steps are needed, and that it is possible to perform the reaction in one pot and that the intermediate β-hydroxy-α-amino acid need not be isolated. This makes the process of the invention very attractive from a commercial and operational point of view.

In the framework of the invention with enantiomerically enriched is meant ‘having an enantiomeric excess (e.e.) of either the (R)- or (S)-enantiomer of a compound’. Preferably, the enantiomeric excess is >60%, more preferably >70%, even more preferably >80%, in particular >90%, more in particular >95%, even more in particular >98%, most in particular >99%.

In one embodiment of the invention, the e.e. of the enantiomerically enriched aminoalcohol formed in the process of the invention may be further enhanced by using a resolution procedure known in the art. Resolution procedures are procedures for the separation of enantiomers aimed to obtain an enantiomerically enriched compound. Examples of resolution procedures include crystallization induced resolutions, resolutions via diastereoisomeric salt formation (classical resolutions) or entrainment, chromatographic separation methods, for example chiral simulating moving bed chromatography; and enzymatic resolution.

With a glycine salt is meant a compound consisting of an aminoacetic acid anion and a cation. Examples of cations in a glycine salt include alkalimetal salts, for example sodium; tetravalent N compounds, for example ammonium or tetraalkylammonium, for example tetra butyl ammonium.

Preferably, the aldehyde is of formula 1

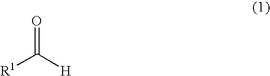

wherein R¹ stands for an optionally substituted (cyclo) alkyl, an optionally substituted (cyclo)alkenyl or an optionally substituted alkynyl, an optionally substituted aryl or for a heterocycle, preferably for an optionally substituted phenyl.

Preferably, the optionally substituted (cyclo) alkyl, the optionally substituted (cyclo)alkenyl or the optionally substituted alkynyl have between 1 and 20 C-atoms, more preferably between 1 and 10 C-atoms (C-atoms of the substituents included).

Alkyls include for example methyl, ethyl, propyl, butyl, pentyl, hexyl, octyl, decyl, isopropyl, sec-butyl, tert-butyl, neo-pentyl and isohexyl. Cycloalkyls include for example cyclopropyl, cyclobutyl, cyclopentyl and cyclohexyl. Alkenyls include for example vinyl, allyl, isopropenyl. (Cyclo)Alkenyls include for example cyclohexenyl and cyclopentadienyl. Alkynyls include for example ethinyl and propynyl.

Preferably, the optionally substituted aryl has between 1 and 20 C-atoms, more preferably between 1 and 10 C-atoms (C-atoms of the substituents included). Optionally substituted aryls include for example: phenyl, naphtyl and benzyl.

Preferably, the optionally substituted heterocycle has between 1 and 20 C-atoms, more preferably between 1 and 10 C-atoms (C-atoms of the substituents included). Heterocycles include for example optionally substituted aromatic heterocycles, for example pyrid-2-yl, pyrid-3-yl, pyrimidin-2-yl, furan-2-yl, furan-3-yl, thiophen-2-yl, imidazol-2-yl, imidazol-5-yl; and optionally substituted (partially) saturated heterocycles, for example morpholin-2-yl, piperidin-2-yl and piperidin-3-yl.

The (cyclo)alkyl, the (cyclo)alkenyl, the alkynyl, the aryl and the heterocycle may be unsubstituted or substituted, and substituents may be substituted in one or more positions. Phenyl may for example be substituted on the ortho and/or meta and/or para position.

Substituents include for example alkyl, for example with 1 to 4 C-atoms; aryl, for example with 3-10 C-atoms; halogens, for example F, Cl, Br, I; borone containing groups, for example B(OH)₂, B(CH₃)₂, B(OCH₃)₂, amines of formula NR²R³, wherein R² and R³ each independently stand for H, alkyl, aryl, OH, alkoxy or for a known N-protection group, for example formyl, acetyl, benzoyl, benzyl, benzyloxy, a carbonyl, an alkyloxycarbonyl, for example t-butyloxycarbonyl, fluoren-9-yl-methoxycarbonyl, sulfonyl, for example a tosyl, or for a silyl, for example trimethylsilyl or tent-butyl diphenylsilyl; isocyanates; an azide; isonitrile; a cyano group; OR⁴, wherein R⁴ stands for H, alkyl, aryl or for a known O-protection group, for example benzyl, acetyl, benzoyl, alkyloxy carbonyl, for example methoxymethyl, silyl, tetrahydropyran-2-yl, sulfonyl, for example tosyl, or for phosphoryl; a (tri-substituted) silyl, for example tri-methyl silyl or tri-phenylsilyl; a phosphorus containing group, for example —P(R⁵)₂, —P(R⁶)₃⁺X⁻, —P(═O)(OR⁷)₂, —P(═O)(R⁸)₂, wherein R⁵, R⁶, R⁷ and R⁸ each independently stand for alkyl, aryl and wherein X— stands for an anion, for example a halogen; nitro, nitroso, SR⁹, wherein R⁹ stands for H, alkyl or aryl; SSR¹⁰, wherein R¹⁰ stands for H, alkyl or aryl; a sulfonic acid (ester) or a salt thereof, for example SO₂ONa, SO₂OCH₃; SO₂R¹¹, wherein R¹¹ stands for alkyl, aryl or H; SOR¹², wherein R¹² stands for alkyl, aryl, or H; SO₂NR¹³R¹⁴, wherein R¹³ and R¹⁴ each independently stand for alkyl, aryl, or H; SeR¹⁵, wherein R¹⁵ stands for alkyl, aryl, or H; SO₂Cl or a heterocyle, for example piperidin-1-yl, morpholin-4-yl, benzotriazol-1-yl, indol-1-yl, pyrrol-1-yl, imidazol-1-yl.

Reacting glycine and the aldehyde of formula 1 in the process of the invention will form the corresponding enantiomerically enriched β-aminoalcohol of formula 2

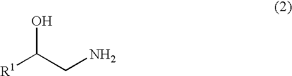

wherein R¹ is as defined above. It is presumed that the reaction proceeds via a β-hydroxy-α-amino acid intermediate of formula (3)

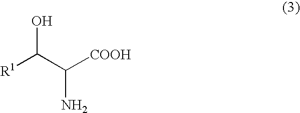

wherein R¹ is as defined above, however, the possibility of another mechanism is not excluded

Preferably, the formed enantiomerically enriched β-aminoalcohol is 2-amino-1-phenylethanol, 2-amino-1-(4-hydroxyphenyl)ethanol, 2-amino-1-(3-hydroxyphenyl)ethanol, 2-amino-1-(3,4-dihydroxyphenyl)ethanol, 2-amino-(4-fluorophenyl)ethanol, 2-amino-(3-fluorophenyl)ethanol2-amino-(2-fluorophenyl)ethanol, 2-amino-(3-chlorophenyl)ethanol. Preferably the formed enantiomerically enriched β-aminoalcohol is a β-aminoalcohol of formula (2), wherein R¹ is as defined above, more preferably a β-aminoalcohol of formula (2) wherein R¹ stands for phenyl, 3-hydroxyphenyl, 4-hydroxyphenyl, 3,4-dihydroxyphenyl, 2,4-dihydroxyphenyl, O,O′-methylene-3,4-dihydroxyphenyl, 3-(hydroxymethyl)-4-hydroxyphenyl, 2-chlorophenyl, 3-chlorophenyl, 4-chlorophenyl, 2-chloro-4-hydroxyphenyl, 4-methoxyphenyl, 2-fluorophenyl, 3-fluorophenyl, 4-fluorophenyl, 2-furanyl, 2-pyridyl, 3-pyridyl, 4-pyridyl, cyclohexyl.

In the framework of the invention, with threonine aldolase is meant an enzyme having threonine aldolase activity, which belong to the group of aldehyde dependent carbon carbon lyases (EC 4.1.2), and preferably belonging to the enzyme classification classes of EC 4.1.2.5 or EC 4.1.2.25, Threonine aldolase activity is defined as the ability to catalyze the reversible splitting of a β-hydroxy-α-amino acid into glycine and the corresponding aldehyde. Threonine aldolases are sometimes also referred to as phenylserine aldolases or β-hydroxy aspartate aldolases. Threonine aldolases are virtually ubiquitous enzymes and may for example be found in Bacteria, Archaea, yeasts and fungi including for example Pseudomonas putida, P. aeruginosa, P. fluorescence, Escherichia coli, Aeromonas jandaei, Thermotoga maritima, Silicibacter pomeroyi, Paracoccus denitrificans, Bordetella parapertussis, Bordetella bronchiseptica, Colwellia psychrerythreae and Saccharomyces cerevisiae. Preferably, a threonine aldolase from a Pseudomonas species, such as e.g. P. putida, P. fluorescence or P. aeruginosa is used. It is known to the person skilled in the art how to find threonine aldolases that are (most) suitable for the conversion of glycine and the specific aldehyde corresponding to the desired intermediate β-hydroxy-α-amino acid leading to the desired β-amino alcohol. More preferably, a threonine aldolase from P. putida is used. Most preferably, a threonine aldolase from P. putida NCIMB12565 or P. putida ATCC12633 is used.

In the framework of the invention, with decarboxylase is meant an enzyme having decarboxylase activity.

Preferably a Carbon-carbon Carboxy Lyase (EC 4.1.1) is used as decarboxylase. More preferably the decarboxylase is an amino acid decarboxylase belonging to aromatic amino acid decarboxylases (EC 4.1.1.28) or a tyrosine decarboxylase (EC 4.1.1.25). In the physiological reaction of tyrosine decarboxylase enzyme, aromatic amino acids such as tyrosine are decarboxylated to an aromatic primary amine such as tyramine and carbon dioxide. Tyrosine decarboxylases may for example be found in Enterococcus, Lactobacillus, Providencia, Pseudomonas, Fusarium, Gibberella, Petroselinum or Papaver. Preferably, a tyrosine decarboxylase from a bacterium belonging to the order of Lactobacillales is used. Even more preferably a tyrosine decarboxylase from Lactobacillus brevis, Enterococcus hirae, Enterococcus faecalis or Enterococcus faecium is used. Most preferably, a tyrosine decarboxylase from Enterococcus faecalis V538, Enterococcus faecalis JH2-2 or Enterococcus faecium DO is used. It is known to the person skilled in the art how to find tyrosine decarboxylases that are (most) suitable for the conversion of the β-hydroxy-α-amino acid leading to the desired β-amino alcohol.

Specifically preferred are decarboxylases having the sequence of [SEQ ID No. 2], [SEQ ID No. 4] or of [SEQ ID No. 6] and homologues thereof. A nucleic acid sequence encoding the decarboxylases of [SEQ ID No. 2], [SEQ ID No. 4] and of [SEQ ID No. 6] is given in [SEQ ID No. 1], [SEQ ID No. 3] or of [SEQ ID No. 5], respectively.

Homologues are in particular decarboxylases having a sequence identity of at least 55%, preferably at least 65%, more preferably at least 70%, more preferably at least 75%, more preferably at least 80%, in particular at least 85%, more in particular at least 90%, at least 91%, at least 92%, at least 93%, at least 94%, at least 95%, at least 96%, at least 97%, at least 98% or at least 99% to [SEQ ID No. 2], [SEQ ID No. 4] and of [SEQ ID No. 6].

For purpose of the present invention, sequence identity is determined in sequence alignment studies using ClustalW, version 1.82 (http://www.ebi.ac.uk/clustalw) multiple sequence alignment at default settings (matrix: Gonnet 250; GAP OPEN: 10; END GAPS: 10; GAP EXTENSION: 0.05; GAP DISTANCES: 8).

Further suitable decarboxylases for the conversion of the β-hydroxy-α-amino acid leading to the desired β-amino alcohol can be found in the group of glutamate decarboxylases (EC 4.1.1.15) and hydroxyglutamate decarboxylases (EC 4.1.1.16). These decarboxylases can for example be found in Bacteria such as Escherichia coli (Umbreit & Heneage, 1953, J. Biol. Chem. 201, 15-20). It is known to the person skilled in the art how to find decarboxylases that are (most) suitable for the conversion of the β-hydroxy-α-amino acid leading to the desired β-amino alcohol. Specifically preferred are decarboxylases having the sequence of [SEQ ID No. 17] or [SEQ ID No. 18] and homologues thereof.

Homologues are in particular decarboxylases having a sequence identity of at least 55%, preferably at least 65%, more preferably at least 70%, more preferably at least 75%, more preferably at least 80%, in particular at least 85%, more in particular at least 90%, at least 91%, at least 92%, at least 93%, at least 94%, at least 95%, at least 96%, at least 97%, at least 98% or at least 99% to [SEQ ID No. 17] or [SEQ ID No. 18].

For example, threonine aldolase and decarboxylase may each independently be present—for example in the form of a dispersion, emulsion, a solution or in immobilized form—as crude enzyme, as a commercially available enzyme, as an enzyme further purified from a commercially available preparation, as an enzyme obtained from its source by a combination of known purification methods, in whole (optionally permeabilized and/or immobilized) cells that naturally or through genetic modification possess threonine aldolase and/or decarboxylase activity, or in a lysate of cells with such activity.

If whole cells are used, preferably the cell has both threonine aldolase and decarboxylase activity. The expression of threonine aldolase and/or decarboxylase in the whole cell may be enhanced using methods known to the person skilled in the art.

It will be clear to the person skilled in the art that use can also be made of mutants of naturally occurring (wild type) enzymes with threonine aldolase and/or decarboxylase activity in the process according to the invention. Mutants of wild-type enzymes can for example be made by modifying the DNA encoding the wild type enzymes using mutagenesis techniques known to the person skilled in the art (random mutagenesis, site-directed mutagenesis, directed evolution, gene shuffling, fusion proteins, for example a fusion protein of threonine aldolase and decarboxylase; etc.) so that the DNA encodes an enzyme that differs by at least one amino acid from the wild type enzyme and by effecting the expression of the thus modified DNA in a suitable (host) cell. Mutants of the threonine aldolase and/or decarboxylase may have improved properties, for example with respect to selectivity for the substrate and/or activity and/or stability and/or solvent resistance and/or pH profile and/or temperature profile. Also, or alternatively, the DNA encoding the wild type enzyme may be modified in order to enhance the expression thereof.

In the framework of the invention, with enantioselective threonine aldolase or enantioselective decarboxylase is meant that the enzyme prefers one of the enantiomers of the β-hydroxy-α-amino acid intermediate corresponding to the aldehyde used, i.e. a threonine aldolase or decarboxylase that has enantioselectivity for either the L- or the D-configuration of the carbon on the position α with respect to the carboxylic acid group (the carbon with an amino group attached). For example, threonine aldolases that are selective for the L-configuration of the carbon a with respect to the carboxylic acid group as well as threonine aldolases that are selective for the D-configuration thereof are known to the person skilled in the art.

Preferably, the enantioselectivity of at least one of the enzymes is at least 90%, more preferably at least 95%, even more preferably at least 98% and most particularly at least 99%.

In the framework of the invention, with a 90% enantioselectivity of for example threonine aldolase is meant that glycine and an aldehyde are converted into 90% of the one enantiomer of the β-hydroxy-α-amino acid intermediate corresponding to the aldehyde used (for example β-hydroxy-L-α-amino acid) and into 10% of the other enantiomer of the corresponding β-hydroxy-α-amino acid (for example β-hydroxy-D-α-amino acid), which corresponds to an enantiomeric excess of 80% of the one enantiomer of the β-hydroxy-α-amino acid (for example β-hydroxy-L-α-amino acid).

Preferably if both the threonine aldolase and the decarboxylase are enantioselective, both the threonine aldolase and the decarboxylase are enantioselective for the same enantiomer of the β-hydroxy-α-amino acid. The higher the enantioselectivity of the threonine aldolase and/or the decarboxylase, the more preferred it is that the threonine aldolase and the decarboxylase enzymes are enantioselective for the same enantiomer of the β-hydroxy-α-amino acid.

In the framework of the invention, with β-selective is meant a threonine aldolase or decarboxylase with a preference (β-selectivity) for one or the other configuration of the β-carbon atom of the β-hydroxy-α-amino acid. In other words, ‘β-selective’ is defined as ‘selective for the configuration of the β-carbon of the intermediate β-hydroxy-α-amino acid’. With β-carbon is meant, the carbon atom in β-position with respect to the carboxylic acid group, i.e. the carbon with the hydroxy group attached.

Preferably, the β-selectivity of at least one of the enzymes is at least 50%, more preferably at least 60%, even more preferably at least 70%, in particular 80%, more in particular at least 90%, even more in particular at least 95%, most in particular at least 99%.

With 90% β-selectivity of threonine aldolase is meant that glycine and an aldehyde are converted by the threonine aldolase into 90% of the one stereoisomer of a β-hydroxy-α-amino acid (for example β-threo-hydroxy-α-amino acid) and into 10% of the other stereoisomer of said β-hydroxy-α-amino acid (for example β-erythro-hydroxy-α-amino acid). The diastereomeric excess (d.e.) of the preferably formed stereoisomer (for example β-threo-hydroxy-α-amino acid) will then be 80%.

With 90% β-selectivity of decarboxylase is meant that if both stereoisomers of a β-hydroxy-α-amino acid are present in equal amounts, decarboxylase, at an overall conversion of 50%, has converted 90% of the one stereoisomer of said β-hydroxy-α-amino acid (for example β-erythro-hydroxy-α-amino acid) and 10% of the other stereoisomer (for example β-threo-hydroxy-α-amino acid).

Preferably if both the threonine aldolase and the decarboxylase are β-selective, both the threonine aldolase and the decarboxylase are β-selective for the same configuration of the β-carbon of the β-hydroxy-α-amino acid. The stronger the β-selectivity of the threonine aldolase and/or the decarboxylase, the more preferred it is that the threonine aldolase and the decarboxylase enzymes are β-selective for the same β-hydroxy-α-amino acid.

In a preferred embodiment of the invention, at least either the threonine aldolase or the decarboxylase is enantioselective.

The reaction conditions chosen depend on the choice of enzyme and the choice of aldehyde. The person skilled in the art known how to optimize various parameters such as temperature, pH, concentration, use of solvent etc.

The temperature and the pH are not very critical in the process of the invention. Preferably, however, the process is carried out at a pH between 4 and 10. In particular, the conversion is carried out at a pH of 4.5 and higher, and at a pH of 6.5 and lower. The temperature is preferably chosen between 0 and 80° C. Preferably, the temperature is higher than 5° C., more preferably higher than 10° C. Preferably the temperature is lower than 50° C., more preferable lower than 39° C.

Suitable solvents for the process of the invention include: water, one phase mixtures of water and a water miscible organic solvent, for example alcohols miscible with water,—for example methanol-, dimethylsulfoxide, dimethylformamide, N-methylpyrrolidone, acetonitrile; or two-phase mixtures of water and a non-miscible organic solvent, for example hydrocarbons, ethers etc; or so-called ionic liquids like, for example, 1,3-dialkyl imidazolium salts or N-alkyl pyridinium salts of acids like hexafluorophosphoric acid, tetrafluoroboric acid, or trifluoromethane sulphonic acid, or with (CF₃SO₂)₂N⁻ as anionic counterpart. Preferably, in the process of the invention a one-phase mixture of water and dimethylsulfoxide (DMSO) is used, for example water with a DMSO content between 1 and 50% v/v, more preferably between 5 and 30% v/v, most preferably between 10 and 20% v/v.

Also, it is possible to perform the process of the present invention in an emulsion system, such as macro- or micro-emulsions, bi-continuous systems comprising an organic phase (with aldehyde substrate), an aqueous phase (usually glycine or a glycine salt, with threonine aldolase and decarboxylase) and a suitable surfactant (non-ionic, cationic or anionic) and the like.

For purpose of the present invention, an emulsion system is defined as a ternary mixture of water, a surfactant and an oil phase, which may be an aliphatic alkane. Examples of aliphatic alkanes which may be used as oil phase in an emulsion include: cyclohexane, isooctane, tetradecane, hexadecane, octadecane, squalene. Surfactants can be any non-ionic, cationic or anionic surfactant, for example Triton X-100, sodium dodecyl-sulfate, AOT, CTAB, Tween-80, Tween-20, Span-80 etc. An oil-in-water (O/W) emulsion may for instance be formed by intense mixing which leads to an increased internal surface and thus facilitates mass transfer between the phases. Especially interesting emulsions are microemulsions that are thermodynamically stable and have a domain size in the nanometer range (see for instance Clapés et al., Chem. Eur. J. 2005, 11, 1392-1401 and Schwuger et al., Chem. Rev. 1995, 95, 849-864).

The molar ratio between glycine or a salt thereof and the aldehyde is in principle not critical. Preferably the molar ratio between glycine or a salt thereof and the aldehyde is >1 and may for example be 1000:1, preferably 100:1, more preferably 10:1.

The order of addition of the reagents, glycine or a salt thereof and the aldehyde; and the enzymes, decarboxylase and threonine aldolase is in principle not critical. For example, the process may be conducted in batch (i.e. everything added at once) or in a fed-batch mode (typically i.e. by feeding one or both reagents; however, enzyme(s) may also be fed). It may be of advantage to remove the β-amino alcohol formed during the reaction and/or to recycle threonine aldolase and/or to recycle decarboxylase. This can be done in between batches, but may of course also be done continuously.

It may be of preference to add cofactors to the reaction to enhance the enzymatic activity of threonine aldolase and/or decarboxylase. Examples of cofactors are known to the person skilled in the art and include pyridoxal-5-phosphate, coenzyme B12, flavin adenine dinucleotide, phosphopantheine, thiamine, S-adenosylmethionine, biotin, salts, for example Mg²⁺, Mn²⁺, Na⁺, K⁺ and Cl⁻. For example, pyridoxal-5-phosphate may be added to the process, for example in a concentration between 0.001 and 10 mM, preferably between 0.01 and 1 mM, more preferably between 0.1 and 0.5 mM. The selection of cofactor depends on the selection of enzyme, for example the enzymatic activity of tyrosine decarboxylase from Enterococci and threonine aldolase from P. putida may be enhanced by addition of pyridoxal-5-phosphate.

The amount of threonine aldolase and/or decarboxylase is in principle not critical. Optimal amounts of threonine aldolase and/or decarboxylase depend on the substrate aldehyde used and can easily be determined by the person skilled in the art through routine experimentation.

The concentration of glycine or a salt thereof used is in principle not critical. Preferably glycine or a salt thereof is used in a concentration between 0.1 and 4 M, more preferably between 0.5 and 3 M, most preferably between 1.0 and 2.5 M.

The concentration of aldehyde is in principle not critical. Preferably the aldehyde is used in a concentration between 1 and 1000 mM, more preferably between 10 and 500 mM, most preferably between 20 and 100 mM.

The product obtained by the process according to the invention may be a pharmaceutical product, for example Noradrenalin or Norfenefrine.

In a further aspect, the invention relates to a process wherein the β-amino alcohol formed in the process of any one of claims 1-12 is further converted into an active pharmaceutical ingredient. For example, the process according to the invention may further comprise converting the amine-group of the product obtained by the process according to the invention into a tert-butyl protected amine group. For example, levabuterol may be obtained in this way. It is also possible that the process according to the invention further comprises converting the amine group of the product obtained by the process according to the invention inte an iso-propyl protected amine group. For example, Sotalol may be obtained this way.

The invention will now be elucidated by way of the following examples without however being limited thereto.

### EXAMPLES

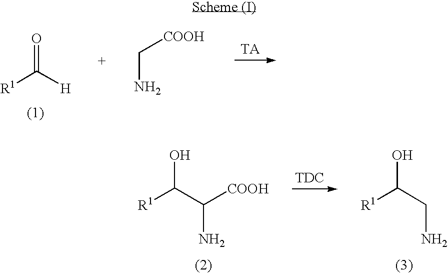

Scheme (I) is meant to illustrate the examples. Scheme (I) is not meant to limit the invention in any way. In Scheme (I) an aldehyde of formula (1) wherein R¹ is as described above is reacted with glycine in the presence of threonine aldolase (TA) to form the corresponding β-hydroxy-α-amino acid intermediate of formula (2) which is then converted in the presence of decarboxylase (TDC) into the corresponding β-aminoalcohol of formula (3). By using a β-selective threonine aldolase or a β-selective decarboxylase, the β-aminoalcohol of formula (3) will be enantiomerically enriched.

### Cloning of L-Tyrosine Decarboxylase Genes

Three open reading frames (ORFs) potentially encoding for three L-tyrosine decarboxylases (TyrDCs) from two Enterococcus species were cloned using the Gateway cloning system (Invitrogen): The tyrD gene of Enterococcus faecalis V583 [SEQ ID No. 1] encoding tyrosine decarboxylase (EfaTyrDC) with the amino acid sequence as given in [SEQ ID No. 2] and further two ORFs with high identities to the E. faecalis V583 tyrD gene, which were identified in the genome sequence of Enterococcus faecium DO. The E. faecium DO tyrD1 gene [SEQ ID No. 3] is 78% identical to the DNA sequence of tyrD from E. faecalis V583 [SEQ ID No. 1] and the corresponding amino acid sequence of EfiTyrDC-1 [SEQ ID No. 4] is 83% identical to the amino acid sequence of EfaTyrDC [SEQ ID No. 2]. The E. faecium DO tyrD2 gene [SEQ ID No. 5] is 62% identical to the DNA sequence of tyrD from E. faecalis V583 [SEQ ID No. 1] and the corresponding amino acid sequence of EfiTyrDC-2 [SEQ ID No. 6] is 59% identical to the amino acid sequence of EfaTyrDC [SEQ ID No. 2]. The two L-tyrosine decarboxylase genes from E. faecium DO share 63% identity on the DNA level [SEQ ID No. 3+5] and 59% identity of the corresponding amino acid sequences [SEQ ID No. 4+6].

Six gene specific primers [SEQ ID No. 7-12] containing attB sites suitable for Gateway cloning (Invitrogen) by homologous recombination were developed for the three L-tyrosine decarboxylase genes [SEQ ID No. 1, 3+5] and synthesized at Invitrogen (UK). These primers were used in at least 3 independent PCR reactions for each gene, respectively, with previously isolated genomic DNA of E. faecalis V583 and E. faecium DO as template, respectively. Proofreading Supermix HiFi DNA polymerase (Invitrogen) was used to amplify tyrD and tyrD1 according to the supplier's procedure with an annealing temperature of 48° C. for tyrD from E. faecalis V583 and 44° C. for tyrD1 from E. faecium DO. For the amplification of tyrD2 from E. faecium DO the proofreading Platinum Pfx DNA polymerase (Invitrogen) was used at 54° C. annealing temperature. For all PCRs only specific amplification products of the expected size of about 1,900 base pairs (bp) were obtained. The tyrD, tyrD1 and tyrD2 amplification products were pooled and purified (QiaQuick PCR purification kit, Qiagen), respectively.

The purified PCR products were used in the Gateway BP cloning reactions to insert the target genes into the intermediate cloning vector pDONR201 (Invitrogen) generating the respective entry vectors pENTR-tyrD, pENTR-tyrD1, and pENTR-tyrD2. After transformation of competent Escherichia coli DH5α cells (Invitrogen), the resulting transformands were pooled and the total plasmid DNA was isolated (Plasmid DNA Spin Mini Kit, Qiagen).

The pool plasmid preparations of pENTR-tyrD, pENTR-tyrD1, and pENTR-tyrD2 were analyzed by restriction analysis with restriction enzymes specific for each gene. From the restriction patterns it could be concluded than ≧99% of the pool plasmid preparations contained the expected fragments. The plasmids pENTR-tyrD, pENTR-tyrD1, and pENTR-tyrD2 were then applied in the Gateway LR cloning reactions with the plasmid pDEST14 (Invitrogen) to obtain the expression vectors pDEST14-tyrD_Efa, pDEST14-tyrD1_Efi, and pDEST14-tyrD2_Efi, respectively. The transformation of E. coli TOP10 with the LR reactions yielded more than hundred individual colonies, respectively. Three clones per gene were tested by restriction analysis and it was found that they gave the expected restriction patterns, respectively.

Heterologous Expression of L-Tyrosine Decarboxylase Genes in Escherichia coli

The isolated pDEST14 expression plasmids were used for the transformation of chemically competent E. coli BL21(DE)pLysS cells, which were subsequently plated on selective Luria-Bertani medium (LB plus 100 μg/ml carbenicillin and 35 μg/ml chloramphenicol). Three to four single colonies were used to inoculate 50 ml precultures (LB plus 100 μg/ml carbenicillin and 35 μg/ml chloramphenicol) for each of the three L-tyrosine decarboxylase genes from the two Enterococcus species. The precultures were incubated on a gyratory shaker at 180 rotations per minute (rpm) at 28° C. over night. Out of these precultures three 1 l LB cultures (supplemented with 100 μg/ml carbenicillin and 35 μg/ml chloramphenicol) were inoculated to cell densities of approximately OD₆₂₀=0.05. These expression cultures were then incubated on a gyratory shaker at 180 rpm and 28° C. The expression of the three target tyrD genes was induced in the middle of the logarithmic growth phase (OD₆₂₀ of about 0.6) by addition of 1 mM isopropyl-β-D-thio-galactoside (IPTG) to the respective cultures. The incubation was continued under the same conditions for four hours. Subsequently the cells were harvested by centrifugation (10 min at 5,000×g, 4° C.) and resuspended in 50 ml of a citrate/phosphate buffer pH 6.0 (0.037 M citric acid+0.126 M Na₂HPO₄) containing 100 μM pyridoxal 5′-phosphate (PLP) and 1 mM dithiothreitol (DTT), respectively. The cell suspensions were frozen at −85° C. To lyse the cells and obtain the cell free extracts, the suspensions were thawed in a 30° C. water bath, subsequently incubated on ice for one hour and centrifuged (30 min at 39,000×g, 4° C.) to remove the cell debris. The supernatants were transferred to new flasks (cell free extracts).

Tyrosine Decarboxylase Activity Assay with DL-Threo-Phenylserine

The tyrosine decarboxylase activity in cell free extracts containing overexpressed TyrDC from E. faecalis V583, E. faecium DO TyrDC-1 or TyrDC-2 was determined with DL-threo-phenylserine as substrate. 0.9 ml of 100 mM of DL-threo-phenylserine (Sigma-Aldrich) solution in citrate/phosphate buffer pH 5.5 (0.043 M citric acid+0.114 M Na₂HPO₄) containing 100 μM PLP and 1 mM DTT was incubated with 0.1 ml cell free extract at room temperature (25° C.). At regular time intervals 50 μl samples were withdrawn and stopped with 950 μl of 0.1 M HClO₄ (in water, pH 1). The decrease of L-threo-phenylserine and the formation of (R)-2-amino-1-phenyl-ethanol was quantified by HPLC on a Crownether Cr(+) column (Daicel) using commercial DL-threo-phenylserine, (R)-2-amino-1-phenyl-ethanol and (S)-2-amino-1-phenyl-ethanol (Sigma-Aldrich) as reference material and a wavelength of 206 nm for detection of substrate and product. One U of tyrosine decarboxylase activity is defined as the amount of enzyme required for the decarboxylation of 1 μmol DL-threo-phenylserine to 2-amino-1-phenyl-ethanol in one minute at 25° C. in citrate/phosphate buffer pH 5.5 (0.043 M citric acid+0.114 M Na₂HPO₄) containing 100 μM PLP and 1 mM DTT.

For Escherichia coli cell free extracts with overexpressed TyrDC from Enterococcus faecalis V583 and TyrDC-1 from E. faecium DO specific activities of 150-160 U/g total protein in the cell free extract were obtained. TyrDC-2 from E. faecium DO had a specific activity of about 10 U/g total protein. The HPLC analyses further showed, that all three TyrDCs decarboxylated exclusively the L-form of threo-phenylserine and enantioselectively formed (R)-2-amino-1-phenyl-ethanol only.

Cloning of Threonine Aldolase Gene from Pseudomonas putida NCIMB12565

The Ita gene [SEQ ID No. 13] encoding the low-specificity L-threonine aldolase (L-TA) as given in [SEQ ID No. 14] was obtained from the genomic DNA of the Pseudomonas putida NCIMB12565 strain by PCR amplification using gene specific primers [SEQ ID No. 15+16]. The PCR reaction was carried out in 50 μl Pfx amplification buffer (Invitrogen), 0.3 mM dNTP, 1 mM MgSO₄, 15 pmol of each primer, 1 μg of genomic DNA, and 1.25 units of the proofreading Platinum Pfx DNA polymerase (Invitrogen). Temperature cycling was as follows: (1) 96° C. for 5 min; (2) 96° C. for 30 sec, 46.7° C. for 30 sec, and 68° C. for 1.5 min during 5 cycles; (3) 96° C. for 30 sec, 51.7° C. for 30 sec, and 68° C. for 1.5 min during 25 cycles.

The forward primer contains an ATG start codon and reverse primer contains a TCA stop codon. BsmBI restriction sites were introduced to obtain PCR fragments with NcoI and XhoI compatible overhangs. The amplified fragment was digested with BsmBI and ligated into pBAD/Myc-His C vector (Invitrogen), which was digested with NcoI and XhoI. The resulting construct pBAD/Myc-His C_LTA_pp 12565 was used to transform E. coli TOP10 cells.

Heterologous Expression of the Ita Gene in Escherichia coli

The recombinant E. coli cells containing pBAD/Myc-HisC_LTA_pp 12565 were precultivated overnight at 28° C. in 50 ml Luria-Bertani medium containing 100 μg/ml carbenicillin. The precultures were used to inoculate 1 l of the same medium containing 100 μg/ml carbenicillin and grown at 28° C. with shaking at 200 rpm. At an OD₆₂₀ of 0.5-1, the cells were induced by adding 0.002% (w/v) L-arabinose. The cells were further incubated over night at room temperature (20-22° C.) with shaking at 200 rpm. The cells were harvested by centrifugation at 12,500×g for 15 min and washed twice with 50 mM TrisHCl buffer (pH 7.5) containing 10 μM PLP and 10 mM DTT. After resuspension of the cells in 40 ml of the same buffer, the cells were disrupted by sonification in a MSE Soniprep 150 at 4° C. for 12 min (maximal amplitude, 10 sec on/10 sec off). Cell debris was removed by centrifugation at 20,000×g for 20 min at 4° C. Aliquots of cell free extracts were stored at −20° C. until further use.

Threonine Aldolase Assay with L-Threonine

Activity of the cell free extracts with overexpressed threonine aldolase was determined spectrophotometrical via NADH consumption at room temperature. 50 μL of the CFE (or suitable dilutions thereof) were diluted into 2950 μL of a buffer containing 100 mM HEPES buffer, pH 8, 50 μM pyridoxal 5-phosphate, 200 μM NADH, 30 U of yeast alcohol dehydrogenase (Sigma-Aldrich), and 50 μM L-threonine in a 3 ml glass cuvette (pathlength 1 cm). In this assay L-threonine is converted to acetaldehyde and glycine by the action of the L-threonine aldolase. The acetaldehyde in turn is reduced to ethanol by the yeast alcohol dehydrogenase, which is connected to the oxidation of an equimolar amount of NADH consumption. The NADH consumption was measured as decrease of absorbance at 340 nm in a Perkin-Elmer Lambda 20 spectrophotometer. One U of threonine aldolase activity is defined as the amount of enzyme necessary to split one μmol of L-threonine into glycine and acetaldehyde in one minute in 100 mM HEPES buffer, pH 8 containing 50 μM pyridoxal 5′-phosphate, 200 μM NADH, 30 U of yeast alcohol dehydrogenase (Sigma-Aldrich), and 50 mM L-threonine at room temperature.

For Escherichia coli cell free extracts with overexpressed threonine aldolase from Pseudomonas putida NCIMB12565 specific activities of 18 U/mg total protein in the cell free extract were obtained with L-threonine as substrate. With D-threonine (Sigma-Aldrich) no conversion was obtained.

Threonine Aldolase Assay with DL-Threo-Phenylserine

To compare the applied threonine aldolase and tyrosine decarboxylase amounts in the two-enzyme/one-pot reactions with each other a second activity assay for threonine aldolase with DL-threo-phenylserine was used. 990 μl of a 100 mM of DL-threo-phenylserine (Sigma-Aldrich) solution in citrate/phosphate buffer pH 5.5 containing 100 μM PLP and 1 mM DTT was incubated in a 1 ml quartz cuvette in a Perkin-Elmer Lambda 20 spectrophotometer at room temperature with 10 μl of cell free extract containing overexpressed threonine aldolase from P. putida NCIMB12565. The amount of DL-threo-phenylserine converted to glycine and benzaldehyde by threonine aldolase was quantified as increase of the absorbance at 279 nm using the molar absorption coefficient of benzaldehyde ε₂₇₉=1.4 cm²/μmol. One unit of threonine aldolase activity with the substrate DL-threo-phenylserine is defined as the amount of enzyme necessary to convert 1 μmol of this substrate into benzaldehyde and glycine in one minute under the above described conditions.

For Escherichia coli cell free extracts with overexpressed threonine aldolase from Pseudomonas putida NCIMB12565 specific activities of 10 U/mg total protein in the cell free extract were obtained with DL-threo-phenylserine as substrate at pH 5.5.

### Determination Protein Concentrations in Solution

The concentrations of proteins in solutions such as cell free extracts were determined using a modified protein-dye binding method as described by Bradford in Anal. Biochem. 72, 248-254 (1976).

### Example 1

### Enzymatic synthesis of (R)-2-amino-1-phenyl-ethanol

For the synthesis of enantiomerically enriched (R)-2-amino-1-phenyl-ethanol (R-APE) 0.106 g benzaldehyde was dissolved in 2.3 ml dimethylsulfoxide (DMSO) and mixed with 3.75 g glycine together with 175 U threonine aldolase from P. putida NCIMB12565 (activity assayed on DL-threo-phenylserine) and 22.5 U TyrDC-1 (Tyrosine decarboxylase-1) from Enterococcus faecium DO (activity assayed on DL-threo-phenylserine) in citrate/phosphate buffer pH 6.0 (0.037 M citric acid+0.126 M Na₂HPO₄). The mixture was incubated in a 50 ml round-bottom flask with stirring at room temperature.

At different points in time 50 μl samples were taken and quenched by addition of 950 μl 0.1 M HClO₄ (in water, pH 1) and analyzed on a Daicel Crownether Cr(+) column for the formation of enantiomerically enriched phenylserine and 2-amino-1-phenyl-ethanol (APE) with DL-threo-phenylserine, DL-erythro-phenylserine, (R)-2-amino-1-phenyl-ethanol, and (S)-2-amino-1-phenyl-ethanol as reference materials using a UV detector at 206 nm. The results of the HPLC analyses of this time course experiment are shown in table 1. These results show, that although the threonine aldolase reactions occurs with a maximum diastereomeric excess (d.e.) of only 25%, the coupling with the TyrDC reaction leads to the product (R)-APE with enantiomeric excess (e.e.) of more than 60%. Furthermore the maximum yield of classical dynamic resolutions of 50% is clearly exceeded.

TABLE 1
Conversion of benzaldehyde and glycine by threonine
aldolase and tyrosine decarboxylase to the phenylserine intermediates and 2-amino-1-
phenyl-ethanol products.

| | L- | | | | | | |
|---|---|---|---|---|---|---|---|
| | threo- | | | | | | |
| | phenyl- | L-erythro- | | | e.e. | | d.e. |
| time | serine | phenylserine | (R)-APE | (S)-APE | (R)-APE | Conversion | threo/erythro |
| [h] | [mM] | [mM] | [mM] | [mM] | [%] | [%] | [%] |
| 0 | 0 | 0.2 | 0.2 | 0 | | 0 | |
| 0.5 | 8.6 | 5.1 | 0.2 | 0 | | 0 | 25 |
| 1 | 16.0 | 10.7 | 0.5 | 0.1 | 74 | 1 | 20 |
| 17 | 8.9 | 8.2 | 15.0 | 3.7 | 61 | 37 | 4 |
| 21 | 7.0 | 6.7 | 16.9 | 4.0 | 62 | 42 | 2 |
| 25 | 5.7 | 5.7 | 18.5 | 4.4 | 62 | 46 | 0 |
| 89 | 0 | 0.3 | 24.6 | 6.1 | 61 | 61 | |
| 97 | 0 | 0.3 | 24.5 | 6.1 | 60 | 61 | |

### Example 2

### Enzymatic Synthesis of D-Noradrenalin

For the synthesis of enantiomerically enriched D-noradrenalin (═(S)-2-amino-1-(3,4-dihydroxy-)phenyl-ethanol) 0.138 g 3,4-dihydroxy-benzaldehyde was dissolved in 2.3 ml dimethylsulfoxide (DMSO) and mixed with 3.75 g glycine together with 175 U threonine aldolase from P. putida NCIMB12565 (activity assayed on DL-threo-phenylserine) and 43.8 U TyrDC-1 from Enterococcus faecium DO (activity assayed on DL-threo-phenylserine) in citrate/phosphate buffer pH 6.0 (0.037 M citric acid+0.126 M Na₂HPO₄). The mixture was incubated in a 50 ml round-bottom flask with stirring at room temperature.

At different points in time 50 μl samples were taken and quenched by addition of 950 μl 0.1 M HClO₄ (in water, pH 1) and analyzed on a Daicel Crownether Cr(+) column for the decrease of 3,4-dihydroxy-benzaldehyde and the formation of enantiomerically enriched noradrenalin using a UV detector at 206 nm. The configuration of the produced noradrenalin was determined using commercial DL-noradrenalin and L-noradrenalin (Sigma-Aldrich) as reference material. The results of the HPLC analyses of this time course experiment are shown in table 2.

TABLE 2
Conversion of 3,4-dihydroxy-benzaldehyde and glycine by
threonine aldolase and tyrosine decarboxylase to noradrenalin.

| | | | e.e. | | con- |
|---|---|---|---|---|---|
| | (R)- | (S)- | (S)- | 3,4-dihydroxy- | ver- |
| time | noradrenalin | noradrenalin | noradrenalin | benzaldehyde | sion |
| [h] | [mM] | [mM] | [%] | [mM] | [%] |
| 0 | 0.01 | 0.1 | 71 | 41.4 | 0 |
| 0.5 | 0.01 | 0.1 | 82 | 40.1 | 3 |
| 1 | 0.25 | 0.8 | 81 | 38.5 | 7 |
| 17 | 1.31 | 11.8 | 80 | 27.2 | 34 |
| 21 | 1.67 | 15.3 | 80 | 24.5 | 41 |
| 25 | 1.92 | 17.6 | 80 | 21.3 | 49 |
| 89 | 4.04 | 33.9 | 79 | 3.6 | 91 |
| 97 | 4.11 | 33.3 | 78 | 2.9 | 93 |

### Example 3

### Enzymatic Synthesis of (S)-Octopamine

For the synthesis of enantiomerically enriched (S)-octopamine (═(S)-2-amino-1-(4-hydroxy-)phenyl-ethanol) 0.977 g 4-hydroxy-benzaldehyde was dissolved in 16 ml dimethylsulfoxide (DMSO) and mixed with 30 g glycine together with 1,400 U threonine aldolase from P. putida NCIMB12565 (activity assayed on DL-threo-phenylserine) and 40 U TyrDC-1 from Enterococcus faecium DO (activity assayed on DL-threo-phenylserine) in citrate/phosphate buffer pH 6.0 (0.037 M citric acid+0.126 M Na₂HPO₄). The mixture was incubated in a 250 ml round-bottom flask with stirring at room temperature.

At different points in time 50 μl samples were taken and quenched by addition of 950 μl 0.1 M HClO₄ (in water, pH 1) and analyzed on a Daicel Crownether Cr(+) column for the formation of enantiomerically enriched octopamine with commercial (RS)-octopamine (Sigma-Aldrich) as reference material using a UV detector at 206 nm. The results of the HPLC analyses of this time course experiment are shown in table 3.

TABLE 3
Conversion of 4-hydroxy-benzaldehyde and glycine by
threonine aldolase and tyrosine decarboxylase to octopamine.

| | (R)- | | | e.e. | |
|---|---|---|---|---|---|
| | octo- | (S)- | 4-hydroxy- | (S)- | |
| time | pamine | octopamine | benzaldehyde | octopamine | conversion |
| [h] | [mM] | [mM] | [mM] | [%] | [%] |
| 0.5 | 0.1 | 1.9 | 40.0 | 93 | 1 |
| 1 | 0.3 | 6.0 | 32.6 | 89 | 19 |
| 17 | 4.8 | 44.9 | 0.4 | 81 | 99 |
| 21 | 4.9 | 43.3 | 0.3 | 80 | 99 |
| 25 | 4.7 | 42.6 | 0.3 | 80 | 99 |
| 89 | 4.6 | 40.5 | 0.3 | 80 | 99 |
| 97 | 4.4 | 41.1 | 0.4 | 81 | 99 |

The reaction mixture was acidified to pH 1-2, and precipitated protein was removed by centrifugation. After titration to pH 3 an ultrafiltration was applied (Amicon 8050 stirred cell, YM-10 membrane, Millipore). The ultrafiltrate was concentrated to 0.1 l in vacuo, acetone was added, and the mixture was stored at −20° C. for 1 h. Precipitated glycine was filtered off, and the filtrate was concentrated to a volume of 40 ml. After adjusting to pH 10.5 with aq. NaOH (30%), the solution was evaporated at 60° C. in vacuo, leaving a liquid residue that was treated with ethyl acetate. Precipitated solids were filtered off, and the filtrate was evaporated in vacuo again. The remaining liquid was purified by column chromatography on 50 g silica with dichloromethane/methanol/25% aq. NH₃ in a ratio of 75/20/5 (v/v/v) as eluent. Fractions containing pure product were pooled and evaporated to give 574 mg (47%) solid (S)-octopamine, identical to an authentic sample by NMR- and HPLC-analysis.

The optical rotation of the product, measured in a Perkin-Elmer 241 polarimeter, was [α]^{D}₂₀=+27.7 (c=0.55, water). The optical rotation reported for (R)-octopamine is [α]^{D}₂₀=+37.4 (c=0.1, water) (Tetrahedron Asymmetry, 2002, Vol. 13, pp. 1209-1217). This corresponds to an e.e. of 74% for the here synthesized (S)-octopamine, which is in agreement with the e.e. value determined by chiral HPLC analysis of 81%.

The NMR data of the (S)-octopamine product are given below: ¹H-NMR (300 MHz, D₂O/DCl, 1,4-dioxane as internal standard (3.75 ppm)): δ 7.3 (m, 2H), 6.95 (m, 2H), 4.96 (dd, 1H), 3.20-3.33 (m, 2H).

¹³C-NMR (75 MHz, D₂O/DCl, 1,4-dioxane as internal standard (67.2 ppm)): δ 156.4, 132.0, 128.4, 116.3, 69.9, 45.9.

### Example 4

### Conversion of DL-Erythro-Phenylserine

Racemic DL-erythro-phenylserine was synthesized according to a procedure as described in EP0220923. DL-erythro-phenylserine was incubated at concentrations of 9 and 5 mM, respectively, with 0.06 U TyrDC-1 from E. faecium DO or 0.18 U TyrDC from E. faecalis V583, respectively, in a total volumes of 1 ml. The reactions were incubated at 25° C. 50 μl samples were taken in the course of the reactions, quenched by addition of 950 μl 0.1 M HClO₄ (in water, pH 1) and analyzed on a Daicel Crownether Cr(+) column for the formation of enantiomerically enriched phenylserine and 2-amino-1-phenyl-ethanol (APE) with DL-threo-phenylserine, DL-erythro-phenylserine, (R)-2-amino-1-phenyl-ethanol, and (S)-2-amino-1-phenyl-ethanol as reference materials using a UV detector at 206 nm. The results of the HPLC analyses are shown in table 4. Neither (R)-2-amino-1-phenyl-ethanol nor D- or L-threo-phenylserine could be detected in any of the samples (detection limits ≦0.004 mM). The concentrations of D-erythro-phenylserine remained constant, while L-erythro-phenylserine decreased over time, indicating that the TyrDCs are enantioselective for the α-amino position.

TABLE 4
Conversion of DL-erythro-phenylserine by EfaTyrDC and
EfiTyrDC-1

| | conversion | | | |
|---|---|---|---|---|
| | DL-erythro- | | d.e. | |
| | phenylserine [%] | | D-erythro-phenylserine[%] | |
| | EfaTyrDC | EfiTyrDC-1 | EfaTyrDC | EfiTyrDC-1 |
| 5 mM after 24 h | 47.6 | 44.9 | 90.8 | 81.6 |
| 5 mM after 42 h | 49.7 | 49.5 | 98.8 | 98.0 |
| 9 mM after 24 h | 45.4 | 41.8 | 83.2 | 71.6 |
| 9 mM after 42 h | 49.7 | 48.7 | 98.7 | 95.0 |

### Example 5

### Conversion of 3,4-dihydroxy-benzaldehyde with and without TyrDC

To 80 μl 0.25 M sodium phosphate buffer pH 6.0 containing 0.1 mM PLP and 2.5 M glycine was added 20 μl of 0.5-1.0 M 3,4-dihydroxy-benzaldehyde (3,4-OH-BA) solution in DMSO. The reaction was started by addition of 0.6 U threonine aldolase from P. putida NCIMB12565 (assayed on DL-threo-phenylserine) and 0.4 U tyrosine decarboxylase from E. faecalis V583 or 0.65 U tyrosine decarboxylase from E. faecium DO (assayed on DL-threo-phenylserine), respectively. In parallel a reaction without tyrosine decarboxylase was set up as a control. All reactions (total volume 0.2 ml) were stirred for 48 hours at room temperature. 25 μl samples were taken in the course of the reactions, quenched by addition of 425 μl 0.1 M HClO₄ (in water, pH 1) and analyzed on a Daicel Crownether Cr(+) column for the decrease of 3,4-dihydroxy-benzaldehyde and the formation of 3,4-dihydroxy-phenylserine (3,4-OH—PS) and enantiomerically enriched noradrenalin using a UV detector at 206 nm. The configuration of the produced noradrenalin was determined using commercial DL-noradrenalin and L-noradrenalin (Sigma-Aldrich) as reference material. The results of the HPLC analyses of this time course experiment are shown in table 5.

It is visible, that without the addition of tyrosine decarboxylase activity only very low conversion of the starting material 3,4-dihydroxy-benzaldehyde is obtained and the formed 3,4-dihydroxy-phenylserine is formed with low β-selectivity, resulting in a d.e. of below 20% for L-erythro-3,4-dihydroxy-phenylserine. In contrast reactions with tyrosine decarboxylase activity exhibit significantly higher conversions of the starting material 3,4-dihydroxy-benzaldehyde. More than 50% up to nearly quantitative conversions of 92% are obtained when tyrosine decarboxylase was added. Furthermore the β-selectivity is significantly improved from below 20% to around 80%, reflected by the e.e. values for D-noradrenalin of 78 to 84% in the reactions containing a tyrosine decarboxylase.

TABLE 5
Conversion of 3,4-dihydroxy-benzaldehyde by threonine
aldolase with and without addition of TyrDC.

| | | L- | L- | | | d.e. | | |
|---|---|---|---|---|---|---|---|---|
| | | threo- | erythro- | | | L-erythro- | | conv. |
| | | 3,4- | 3,4-OH- | | | 3,4-OH- | e.e. D- | 3,4- |
| | time | OH-PS | PS | L-noradrenaline | D-noradrenalin | PS | noradrenalin | OH-BA |
| reaction | [h] | [mM] | [mM] | [mM] | [mM] | [%] | [%] | [%] |
| EfaTyrDC | 3.5 | n.d. | n.d. | 1.0 | 4.8 | n.a. | 65 | 10 |
| 50 mM 3,4- | 48 | n.d. | n.d. | 5.5 | 47.5 | n.a. | 79 | 87 |
| OH-BA | | | | | | | | |
| EfiTyrDC-1 | 3.5 | n.d. | n.d. | 1.0 | 9.2 | n.a. | 80 | 17 |
| 50 mM 3,4- | 48 | n.d. | n.d. | 4.7 | 53.9 | n.a. | 84 | 92 |
| OH-BA | | | | | | | | |
| EfiTyrDC-1 | 3.5 | n.d. | n.d. | 1.2 | 6.4 | n.a. | 69 | 7 |
| 100 mM 3,4- | 48 | 0.28 | 0.53 | 7.1 | 56.5 | 31 | 78 | 60 |
| OH-BA | | | | | | | | |
| without | 3.5 | 0.05 | 0.07 | 0 | 0 | 17 | n.a. | 0.1 |
| TyrDC | 48 | 1.38 | 2.02 | 0 | 0 | 19 | n.a. | 3 |
| 100 mM 3,4- | | | | | | | | |
| OH-BA | | | | | | | | |

n.d.: not detectable;
n.a.: not applicable

The results shown above illustrate that it is an advantage of the process according to the invention that yields higher than 50% may be obtained than for the enantiomerically pure product, in particular when an aromativ aldehyde is converted and a tyrosine decarboxylase is used in the process according to the invention.

### Example 6

### Alternative Substrates (Substituted Aromatic Aldehydes, cf. Formula (1))

To 0.15 ml 0.27 M sodium phosphate buffer pH 6.0 containing 0.13 mM PLP and 1.27 M glycine was added 20 μl 0.25-0.5 M aldehyde solution in DMSO. The reaction was started by addition of 10 μl threonine aldolase from P. putida NCIMB12565 (cell free extract; 59 U/ml, assayed on DL-threo-phenylserine) and 20 μl tyrosine decarboxylase from E. faecalis V583 (cell free extract; 1.8 U/ml, assayed on DL-threo-phenylserine). The solutions were stirred for 1-3 days at room temperature and formation of the corresponding substituted phenylserines (cf. formula (3)) and β-aminoalcohols (cf. formula (2)) was monitored by thin-layer-chromatography on silica coated glass plates. R_{f} values: β-aminoalcohols at R_{f}=0.6-0.7, substituted phenylserines at R_{f}=0.2-0.3, glycine at R_{f}=0 (eluent: dichloromethane/methanol/25% aq. ammonia 75/20/5 (v/v/v); ninhydrine staining). 2-fluorobenzaldehyde, 3-fluorobenzaldehyde, 4-fluorobenzaldehyde, 2-chlorobenzaldehyde, 3-chlorobenzaldehyde, 4-chlorobenzaldehyde, 3-bromobenzaldehyde, 4-bromobenzaldehyde, 3-methylbenzaldehyde, 4-methylbenzaldehyde, 3-hydroxybenzaldehyde, 3-methoxybenzaldehyde, 3-nitrobenzaldehyde, 3,4-(methylenedioxy)-benzaldehyde, 2-furaldehyde, pyridine-2-carboxaldehyde, pyridine-3-carboxaldehyde, pyridine-4-carboxaldehyde and hexahydrobenzaldehyde were converted by threonine aldolase and tyrosine decarboxylase to the corresponding β-hydroxy-α-amino acid intermediates and the corresponding β-aminoalcohols.

### Example 7

### Enzymatic Synthesis of L-Norfenefrine

For the synthesis of enantiomerically enriched L-norfenefrine (═(R)-2-amino-1-(3-hydroxy-)phenyl-ethanol) 0.1 M 3-hydroxy-benzaldehyde was reacted with 1 M glycine in 1 ml total volume together with 38 U threonine aldolase from P. putida NCIMB12565 (activity assayed on DL-threo-phenylserine) and 0.4 U TyrDC from Enterococcus faecalis V583 (activity assayed on DL-threo-phenylserine) in 50 mM KH₂PO₄ buffer pH 5.5 containing 50 μM pyridoxal 5′-phosphate. The mixture was incubated with stirring at room temperature (25° C.).

The reaction was analyzed on a Daicel Crownether Cr(+) column for the formation of enantiomerically enriched norfenefrine with commercial DL-norfenefrine (Sigma) as reference material using a UV detector at 210 nm. After 24 h 76% of the supplied 3-hydroxy-benzaldehyde was converted to enantiomerically enriched L-norfenefrine with an e.e. of 56%. Optical rotation was measured on a Perkin-Elmer 341 polarimeter:

[α]²⁰_{D} −11.1 (c 1.0 in EtOH); literature value: [α]²⁰_{D} −1.7 (c 5.8 in MeOH)

Also NMR data were consistent with those reported by Lundell et al. (Tetrahedron: Asymmetry 2004, 15, 3723).

### Example 8

### Enzymatic synthesis of enantiomerically enriched halogenated 2-amino-1-phenyl-ethanols

To a solution of halogenated benzaldehyde derivative (0.1 mmol), glycine (1.0 mmol) and pyridoxal 5″-phosphate (50 nmol) in 1.0 ml buffer (KH₂PO₄, 50 mM, pH 5.5) 38 U threonine aldolase from P. putida NCIMB12565 (activity assayed on DL-threo-phenylserine) and 0.4 U tyrosine decarboxylase from Enterococcus faecalis V583 or TyrDC-1 from Enterococcus faecium DO (activity assayed on DL-threo-phenylserine) were added. The reaction mixture was stirred at 25° C.; yield and e.e. were determined by HPLC after 24 and 57 hours.

¹H and ¹³C NMR spectra were recorded on a Varian INOVA 500 (¹H 499.82 MHz, ¹³C 125.69 MHz) or on a Varian GEMINI 200 (¹H 199.98 MHz, ¹³C 50.29 MHz) using the residual peaks of CDCl₃ (¹H: δ 7.26, ¹³C δ 77.0), D₂O (¹H: δ 4.79) or DMSO*d₆ (¹H: δ 2.50, ¹³C δ 40.2) as references. H₂O/D₂O-NMR samples were taken directly from the aqueous solution, diluted with D₂O (1:1) and recorded using H₂O presaturation. Analytical HPLC was carried out with a Hewlett Packard Series 1100 HPLC using a G1315A diode array detector. 2-Amino-1-phenylethanol and its derivatives were analyzed on a Crownpack® Cr (−) (150 mm, 5 μm), column under standard conditions (HClO₄-solution pH 1.0, 114 mM, 1.0 ml/min, 15° C.). Optical rotation was measured on a Perkin-Elmer 341 polarimeter.

TABLE 6
Synthesis of enantiomerically enriched halogenated
2-amino-1-phenyl-ethanol.

| | | | EfiTyrDC-1 | |
|---|---|---|---|---|
| | EfaTyrDC | | yield | |
| Product | yield [%] | e.e. (%)^{[a]} | [%] | e.e. (%)^{[a]} |
| 2-Amino-1-phenylethanol | 91^{[a]} (57 h) | 77 (R) | 49^{[a]} | 41 (R) |
| 2-Amino-1-(2- | 81^{[b]} | >99 (S) | 54^{[b]} | >99 (S) |
| fluorophenyl)ethanol | | | | |
| 2-Amino-1-(3- | 82^{[b]} | 66 (R) | 48^{[b]} | 44 (R) |
| fluorophenyl)ethanol | | | | |
| 2-Amino-1-(4- | 36^{[b]} | 76 (R) | 33^{[b]} | 71 (R) |
| fluorophenyl)ethanol | | | | |
| 2-Amino-1-(2- | <2^{[b]} | n.d. | <2^{[b]} | n.d. |
| chlorophenyl)ethanol | | | | |
| 2-Amino-1-(3- | 52^{[b]} | 28 (R) | 6^{[b]} | 11 (R) |
| chlorophenyl)ethanol | | | | |
| 2-Amino-1-(4- | <2^{[b]} | 43 (S) | <2^{[b]} | 14 (S) |
| chlorophenyl)ethanol | | | | |

^{[a]}determined by HPLC;
^{[b]}determined by ¹H-NMR;
n.d.: not determined. If not indicated reaction time was 24 h.

NMR-Data:

### (R)-2-Amino-1-(3-fluorophenyl)ethanol

¹H-NMR (500 MHz, DSMO) δ 2.56 (dd, 1H, CH—N, J=8.0 Hz, J=13.0 Hz), 2.69 (dd, 1H, CH—N, J=4.0 Hz, 12.5 Hz), 4.48 (dd, 1H, CH—O, J=7.5 Hz, J=4.0 Hz), 7.02 (dt, 1H, ArH,

### J=2.0 Hz, J=8.5 Hz), 7.13 (m, 2H, ArH), 7.33 (dd, 1H, ArH, J=8.0 Hz, J=14.5 Hz);

¹³C-NMR (500 MHz, DMSO* d₆) δ 50.5, 74.3, 113.2 (d, J=21.5 Hz), 114.0 (d, J=21.0 Hz), 122.6 (d, J=2.4 Hz), 130.5 (d, J=8.1 Hz), 148.3 (d, J=6.8 Hz), 162.9 (d, J=241 Hz);

[α]²⁰_{D} −29.3 (c 1.0 in EtOH)

### (R)-2-Amino-1-(4-fluorophenyl)ethanol

[α]²⁰_{D} −12.5 (c 1.0 in EtOH); literature value for (S)-2-Amino-1-(4-fluorophenyl)ethanol

[α]²⁰_{D} +40.9 (c 0.48 in EtOH); HPLC: t_{S}=28.8 min, t_{R}=32.2 min; Also NMR data were consistent with those reported by Cho et al. (Tetrahedron: Asymmetry 2002, 13, 1209).

### (R)-2-Amino-1-(2-chlorophenyl)ethanol

[α]²⁰_{D} −59 (c 1.0 in EtOH); literature value for (S)-2-Amino-1-(2-chlorophenyl)ethanol

[α]²⁰_{D} +92.5 (c 1.02 in CH₂Cl₂); HPLC: t_{S}=21.0 min, t_{R}=24.6 min; Also NMR data were consistent with those reported by Noe et al. (Monatsh. Chem. 1995, 126, 481)

### (R)-2-Amino-1-(3-chlorophenyl)ethanol

[α]²⁰_{D} −28.7 (c 1.0 in EtOH); literature value for (S)-2-Amino-1-(3-chlorophenyl)ethanol

[α]²⁰_{D} +78.9 (c 0.21 in EtOH); HPLC: t_{S}=20.5 min, t_{R}=23.9 min; Also NMR data were consistent with those reported by Cho et al. (Tetrahedron: Asymmetry 2002, 13, 1209).

### (R)-2-Amino-1-(4-chlorophenyl)ethanol

[α]²⁰_{D} −34.4 (c 1.0 in EtOH); literature value for (S)-2-Amino-1-(4-chlorophenyl)ethanol

[α]²⁰_{D} +40.5 (c 0.53 in EtOH); HPLC: t_{S}=20.5 min, t_{R}=23.7 min; Also NMR data were consistent with those reported by Cho et al. (Tetrahedron: Asymmetry 2002, 13, 1209).

### Example 9

### Conversion of Aliphatic Compounds with Threonine Aldolases and Tyrosine Decarboxylase

For the simultaneous one-pot conversion of cyclohexyl-carboxaldehyde and glycine by threonine aldolase and tyrosine decarboxylase to 2-amino-1-cyclohexylethanol 40 U threonine aldolase from P. putida NCIMB12565 and 2.5 U TyrDC from Enterococcus faecalis V583 or 2.5 U TyrDC-1 from Enterococcus faecium DO were reacted with 0.1 M cyclohexyl-carboxaldehyde and 1.0 M glycine in total volumes of 1 ml phosphate buffer (50 mM, pH 5.5, containing 50 μM PLP). As a control reaction only 40 U of threonine aldolase from P. putida NCIMB12565 was reacted with 0.1 M cyclohexyl-carboxaldehyde and 1.0 M glycine in a total volume of 1 ml phosphate buffer (50 mM, pH 5.5, containing 50 μM PLP). The reactions were incubated at 25° C. with magnetic stirring. After 48 hours the reactions were diluted 7.5 times with 0.5% methanesulfonic acid (in water pH 1.3) and analysed by LC-MS using a Prevail C18 column (250×4.0 mm, 5 μm; eluent A: 0.5% methanesulfonic acid in water pH 1.3; eluent B: 0.5% methanesulfonic acid in acetonitrile; flow: 1 ml/min; gradient: 95% eluent A+5% eluent B to 5% eluent A to 95% eluent B within 15 min) coupled with an atmospheric pressure ionisation-electron spray time of flight-MS detector run in positive mode (full scan).

Only in the reactions containing both threonine aldolase and tyrosine decarboxylase 2-amino-1-cyclohexylethanol (retention time 5.65 min) could be identified according to its molecular mass of m+1=144 compared with reference material chemically synthesised according to Mecca et al. (Tetrahedron: Asymmetry 2001, 12, 1225-1233). The control reaction with threonine aldolase only did not result in the formation of detectable amounts of 2-amino-1-cyclohexylethanol, proving that this aliphatic β-aminoalcohol is produced by the coupled threonine aldolase plus tyrosine decarboxylase reactions.

1. Process for the preparation of an enantiomerically enriched β-aminoalcohol, wherein glycine or a glycine salt and an aldehyde are reacted in the presence of a threonine aldolase and a decarboxylase to form the corresponding enantiomerically enriched β-aminoalcohol, and wherein at least either the threonine aldolase or the decarboxylase is β-selective.

2. Process according to claim 1, wherein the decarboxylase is a tyrosine decarboxylase.

3. Process according to claim 1, wherein at least either the threonine aldolase or the decarboxylase is enantioselective.

4. Process according to claim 1, wherein the aldehyde is an aldehyde of formula 1 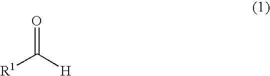
wherein R¹ stands for an optionally substituted (cyclo) alkyl, an optionally substituted (cyclo)alkenyl or an optionally substituted alkynyl, an optionally substituted aryl or for a heterocycle.

5. Process according to claim 1, wherein the β-aminoalcohol is a β-aminoalcohol of formula 2, 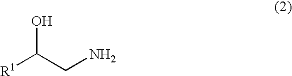
wherein R¹ stands for an optionally substituted (cyclo) alkyl, an optionally substituted (cyclo)alkenyl or an optionally substituted alkynyl, an optionally substituted aryl or for a heterocycle.

6. Process according to claim 5, wherein R¹ stands for phenyl, 3-hydroxyphenyl, 4-hydroxyphenyl, 3,4-dihydroxyphenyl, 2,4-dihydroxyphenyl, O,O′-methylene-3,4-dihydroxyphenyl, 3-(hydroxymethyl)-4-hydroxyphenyl, 2-chlorophenyl, 3-chlorophenyl, 4-chlorophenyl, 2-chloro-4-hydroxyphenyl, 4-methoxyphenyl, 2-fluorophenyl, 3-fluorophenyl, 4-fluorophenyl, 2-furanyl, 2-pyridyl, 3-pyridyl, 4-pyridyl, cyclohexyl.

7. Process according claim 1, wherein the threonine aldolase belongs to the enzyme classification class of EC 4.1.2.5 or EC 4.1.2.25.

8. Process according to claim 1, wherein the decarboxylase belongs to the enzyme classification class of EC 4.1.1.25 or EC 4.1.1.28.

9. Process according to claim 1, wherein the β-selectivity of the threonine aldolase and/or the decarboxylase is at least 50%.

10. Process according to claim 3, wherein the enantioselectivity of the threonine aldolase and/or the decarboxylase is at least 90%.

11. Process according to claim 1, wherein if both the threonine aldolase and the decarboxylase are β-selective, both the threonine aldolase and the decarboxylase are β-selective for the same β-hydroxy-α-amino acid.

12. Process according to claim 3, wherein if both the threonine aldolase and the decarboxylase are enantioselective, both the threonine aldolase and the decarboxylase are enantioselective for the same enantiomer of the β-hydroxy-α-amino acid.

13. Process according to claim 1, wherein the temperature is chosen between 10 and 39° C.

14. Process according to claim 1, further comprising converting the amino-group of the β-amino alcohol formed in the process into a tert-butyl protected amino group.

15. Process according to claim 1, further comprising converting the amino-group of the β-amino alcohol formed in the process into an iso-propyl protected amino group.

16. Process wherein the β-amino alcohol formed in the process of claim 1 is further converted into an active pharmaceutical ingredient.


SEQUENCE LISTING (18 sequences)

SEQ ID NO: 1 - DNA, 1863 bp, Enterococcus faecalis
atgaaaaacgaaaaattagcaaaaggcgaaatgaaccttaatgcactattcattggggacaaagccgaaaacggacaattatataaagacttgttgatcgacttagtagatgaacatttaggatggcgtcaaaactacatgccacaagacatgccagttatctcttctcaagaacgcacatctgaaagctacgaaaaaactgtcaaccatatgaaagatgtattgaatgaaatctcttctcgcatgcgtacacattcagttccatggcatacagcaggtagatattggggacatatgaactcagaaacattaatgccttctctattagcttacaactttgcaatgctatggaacgggaacaacgttgcctatgaatcttctccagcaacttctcaaatggaagaagaagtaggacatgaatttgctcacttaatgagctacaaaaatggttggggacacatcgttgctgatggttctttagctaacttagaaggcttatggtatgcccgtaacattaaatcattaccatttgctatgaaagaagtaaaaccagaattagttgctggcaaatcagattgggaactattgaacatgccaacaaaagaaattatggacttattagaatcagctgaagatgaaattgatgaaatcaaagctcattcagctcgttcaggtaaacatttacaagcaatcggtaaatggttagttccacaaactaaacactattcttggttaaaagctgctgatattatcggtatcggtttagatcaagttatcccagtaccagttgaccataactaccgtatggatatcaacgaattagaaaaaatcgttcgtggcttagcagaagaacaaattccagtgctaggtgttgtcggtgtagttggttctactgaagaaggtgccgttgactcaatcgataaaattattgctttacgcgatgaattaatgaaagacggtatttactattatgtacacgttgatgctgcttatggtggttatggacgtgccatcttcttagacgaagacaacaacttcatcccttacgaagatttacaagatgttcacgaagaatacggtgtcttcaaagagaaaaaagaacacatttcaagagaagtgtatgatgcatataaagcaatcgaattagcagaatcagtaacaattgaccctcataaaatgggttatatcccttattcagctggtggtatcgttatccaagatatccgtatgcgcgacgttatttcttacttcgcaacttacgtatttgaaaaaggtgccgatattcctgcgttactaggtgcttatatccttgaaggttcaaaagctggtgcaacagctgcttctgtatgggctgcacatcacgttttacctttaaacgttgcaggttatggtaaattaattggtgcttcaattgaaggatcacatcatttctataacttcttgaatgatttaacattcaaagttggcgacaaagaaatcgaagtccacacattaactcatccagacttcaacatggttgactatgtgtttaaagaaaaaggcaacgatgatttagtagcaatgaacaaattaaaccatgatgtatatgactatgcatcatacgttaaaggaaatatttacaacaacgaattcattacttcacatactgactttgctattccagattatggcaacagcccattaaaatttgtaaacagcttgggatttagtgacgaagaatggaaccgtgcaggtaaagtaactgtgttacgtgctgctgtaatgactccatacatgaacgataaagaagaatttgatgtttacgctcctaagattcaagctgctttacaagaaaaattagaacaaatttacgacgtaaaataa

SEQ ID NO: 2 - protein, 620 aa, Enterococcus faecalis
MKNEKLAKGEMNLNALFIGDKAENGQLYKDLLIDLVDEHLGWRQNYMPQDMPVISSQERTSESYEKTVNHMKDVLNEISSRMRTHSVPWHTAGRYWGHMNSETLMPSLLAYNFAMLWNGNNVAYESSPATSQMEEEVGHEFAHLMSYKNGWGHIVADGSLANLEGLWYARNIKSLPFAMKEVKPELVAGKSDWELLNMPTKEIMDLLESAEDEIDEIKAHSARSGKHLQAIGKWLVPQTKHYSWLKAADIIGIGLDQVIPVPVDHNYRMDINELEKIVRGLAEEQIPVLGVVGVVGSTEEGAVDSIDKIIALRDELMKDGIYYYVHVDAAYGGYGRAIFLDEDNNFIPYEDLQDVHEEYGVFKEKKEHISREVYDAYKAIELAESVTIDPHKMGYIPYSAGGIVIQDIRMRDVISYFATYVFEKGADIPALLGAYILEGSKAGATAASVWAAHHVLPLNVAGYGKLIGASIEGSHHFYNFLNDLTFKVGDKEIEVHTLTHPDFNMVDYVFKEKGNDDLVAMNKLNHDVYDYASYVKGNIYNNEFITSHTDFAIPDYGNSPLKFVNSLGFSDEEWNRAGKVTVLRAAVMTPYMNDKEEFDVYAPKIQAALQEKLEQIYDVK

SEQ ID NO: 3 - DNA, 1878 bp, Enterococcus faecium DO
atgagtgaatcattgtcgaaagatctaaacctaaatgccctattcattggggacaaagctgagaacggccaaatctataaagctttgttgaatgaattggtggatgaacatttaggctggcgtcaaaactacatgcctcaagacatgcctattattacgccagaagaaaaaagcagtgctagttttgaacacactgtgaataaaacaaaggatgttctatcagaaatttcagcgcggatgcgtacccattccgttccttggcataatgccggacgttactggggtcatatgaactctgaaacattgatgccatctttactagcgtacaattttgctatgctatggaatggtaataacgttgcctatgaatcttctcctgcaacaagccaaatggaagaagaagtaggaatggaatttgcaaaattaatgagctataaagatggctggggccacatcgttgctgatggttctttagcaaacttagaaggactttggtatgcccgcaacatcaaatcattgccgcttgcaatgaaagaagtgacacctgaactagttgctggaaaaagcgattgggaattaatgaacttgtcaacagaagaaatcatgaatctgttagacagcgtcccagaaaaaatcgacgaaatcaaagctcactcagcacgtagcggaaaacatttagaaaaattaggaaaatggttggttccacaaactaaacactattcatggctaaaagctgccgacatcatcggtatcggtttagaccaagtgattccagtgccagttgaccacaactatcgcatggacatcaacgaacttgaaaaaatcgttcgtggtctagcagctgaaaaaacaccaatcctaggtgtagtcggtgttgtaggatcaacagaagaaggagcaatcgacggtatcgataaaatcgttgcattgcgtcgcgtattagaaaaagacggtatttacttctacttacacgtagatgctgcttatggcggatacggccgtgcgattttcttagacgaagacaacaacttcattccatttgaagatttaaaagatgttcattacaaatacaatgtctttacagaaaacaaagattatatcttagaagaagtacacagtgcatataaagcaatcgaagaagcagaatctgtaacgattgacccacataaaatgggctatgttccatattctgccggtggtatcgtcatcaaagatatccgcatgagagacgttatctcttactttgcgacatatgtatttgaaaaaggcgcagatatcccagcattacttggggcttatatcttggaaggttcaaaagcaggggcaacagcagccagcgtttgggcagcacaccatgtcttacctttgaatgtcacaggatatggtaaattaatgggtgcatcaatcgaaggtgcacatcgtttctacaacttcttgaatgacttgtcatttaaagtaggagacaaagaaatcgaagttcatccattaacttatccagatttcaatatggtagactatgtattcaaagaaaaaggcaacgatgacttagttgcaatgaataaattgaaccatgacgtatatgattattcttcatacgtgaaaggaagcatttacggtaatgagttcttaacttcacatactgactttgctatcccagactatggcaacagcccattacaatttgtgaaccaactaggattctcagatgaagaatggaaccgtgccggaaaagtaactgtattgcgtgccagcgtaatgacaccatacatgaacaaagaagaacactttgaagaatatgctgaaaaaatcaaagcagctcttcaagaaaaattggaaaaaatctacgcagaccaattattggcaagcgaagcaaaataa

SEQ ID NO: 4 - protein, 625 aa, Enterococcus faecium DO
MSESLSKDLNLNALFIGDKAENGQIYKALLNELVDEHLGWRQNYMPQDMPIITPEEKSSASFEHTVNKTKDVLSEISARMRTHSVPWHNAGRYWGHMNSETLMPSLLAYNFAMLWNGNNVAYESSPATSQMEEEVGMEFAKLMSYKDGWGHIVADGSLANLEGLWYARNIKSLPLAMKEVTPELVAGKSDWELMNLSTEEIMNLLDSVPEKIDEIKAHSARSGKHLEKLGKWLVPQTKHYSWLKAADIIGIGLDQVIPVPVDHNYRMDINELEKIVRGLAAEKTPILGVVGVVGSTEEGAIDGIDKIVALRRVLEKDGIYFYLHVDAAYGGYGRAIFLDEDNNFIPFEDLKDVHYKYNVFTENKDYILEEVHSAYKAIEEAESVTIDPHKMGYVPYSAGGIVIKDIRMRDVISYFATYVFEKGADIPALLGAYILEGSKAGATAASVWAAHHVLPLNVTGYGKLMGASIEGAHRFYNFLNDLSFKVGDKEIEVHPLTYPDFNMVDYVFKEKGNDDLVAMNKLNHDVYDYSSYVKGSIYGNEFLTSHTDFAIPDYGNSPLQFVNQLGFSDEEWNRAGKVTVLRASVMTPYMNKEEHFEEYAEKIKAALQEKLEKIYADQLLASEAK

SEQ ID NO: 5 - DNA, 1881 bp, Enterococcus faecium DO
atgtatctgcaagacattgaccaacaaaatatggagggaagaaaaatgaaagatatggatatcaaggccgtctttataggcgacaaagctgaaaatggaccagtttataaaatgttgttgaacaagatggtagatgaacatcttggatggagagaaaattatattccttcagatatgcctgcaattagcgaaggagataagctcactccagattatctagccacccgcgaccatatgatagaggtcttggatgaagtcagtcagcgattacgcgcaggatcgatcccatggcattcagcaggccgttactggggacaaatgaatgctgaaacattgatgcctgctttattagcttacaactatgcgatgctgtggaacccaaataatgttgcattggaatcttctatggcaacttcgcaaatggaagcagaagtaggacaggattttgcttcattattcaatatgacagacggatggggacacattgcagcagatggttctattgctaatcttgaaggattatggtacgcccgttgcatcaaatcgattcctttagctgtcaaggaagtgctgcctgaaaaagtgaaaaaaatgtctgagtgggaacttttgaatttatctgttgaagaaattttggaaatgaccgaaagctttacggacgaagaaatggatgaggtcaaagcagcttcttcccgtagcggaaaaaatatccaaagattagggaaatggctggtccctcaaacaaaacactattcttggatgaaggcgttagatatttgcggagtcggcttagatcagatggttgctatcccagttcaagaggattatcgaatggatattaatgctttggaaaaaacaattcgtgaattagctggacaaaaaatcccaattctaggtgtggttgctgtcgtaggaacgacagaagaaggtcaagtagacagtgtagataaaatcgtccaattaagagaaaggttaaaagatgaagggatctatttctatttacatgttgatgccgcatatggcggctatgcgcgttcactgtttctaaacgaagcaggagaatttgtgccgtatgcttccttagctgaattctttgaagaacatcatgttttccaccactgcgtaacgattgacaaagaggtatatgaagggttcagagctatttcagaagcggattctgtcacgatagatcctcataagatgggctatgtcccttatgctgctggaggaatcgtaatcaaacataaaaatatgagaaatatcatttcttatttcgcgccatatgtgtttgaaaaatcagtaaaggctccagatatgttaggagcttatattttggaaggatcaaaagctggagcaaccgccgccgcagtctggacagcacatcgtgtattgccattgaatgtgaccggatatggtcaattgattggggcttcaatcgaggcagctcaaagattcagagaatttctcgatcatctgacttttactgttaaaggaaaaacaatcgaagtctatccattgaatcatcctgattttaatatggtcaattgggtattcaaagaacaaggctgtacagatttgaacgctatcaatgaattaaatgaaaaaatgttcgatcggtcttcttatatggatggcgatgtttatggtgaacggtttattacttctcatactacatttacacaagaagattacggtgattctccgattcgttttgttgaaagaatgggtctaacgaaagaagaatggaaaaaagaacagaaaatcactctgctacgtgcggctatcatgactccttatttgaatgatgatcgaattttcaatttttatacaaaaaaaatcgccaaagcaatggaaaagaaactaaacgaaatcatccaatag

SEQ ID NO: 6 - protein, 626 aa, Enterococcus faecium DO
MYLQDIDQQNMEGRKMKDMDIKAVFIGDKAENGPVYKMLLNKMVDEHLGWRENYIPSDMPAISEGDKLTPDYLATRDHMIEVLDEVSQRLRAGSIPWHSAGRYWGQMNAETLMPALLAYNYAMLWNPNNVALESSMATSQMEAEVGQDFASLFNMTDGWGHIAADGSIANLEGLWYARCIKSIPLAVKEVLPEKVKKMSEWELLNLSVEEILEMTESFTDEEMDEVKAASSRSGKNIQRLGKWLVPQTKHYSWMKALDICGVGLDQMVAIPVQEDYRMDINALEKTIRELAGQKIPILGVVAVVGTTEEGQVDSVDKIVQLRERLKDEGIYFYLHVDAAYGGYARSLFLNEAGEFVPYASLAEFFEEHHVFHHCVTIDKEVYEGFRAISEADSVTIDPHKMGYVPYAAGGIVIKHKNMRNIISYFAPYVFEKSVKAPDMLGAYILEGSKAGATAAAVWTAHRVLPLNVTGYGQLIGASIEAAQRFREFLDHLTFTVKGKTIEVYPLNHPDFNMVNWVFKEQGCTDLNAINELNEKMFDRSSYMDGDVYGERFITSHTTFTQEDYGDSPIRFVERMGLTKEEWKKEQKITLLRAAIMTPYLNDDRIFNFYTKKIAKAMEKKLNEIIQ

SEQ ID NO: 7 - DNA, 69 bp, artificial sequence
  primer
ggggacaagtttgtacaaaaaagcaggctaggaggaattaaccatgaaaaacgaaaaattagcaaaagg

SEQ ID NO: 8 - DNA, 55 bp, artificial sequence
  primer
ggggaccactttgtacaagaaagctgggtgtttaagattcaattattttacgtcg

SEQ ID NO: 9 - DNA, 68 bp, artificial sequence
  primer
ggggacaagtttgtacaaaaaagcaggctaggaggaattaaccatgagtgaatcattgtcgaaagatc

SEQ ID NO: 10 - DNA, 54 bp, artificial sequence
  primer
ggggaccactttgtacaagaaagctgggtccttgcttattttgcttcgcttgcc

SEQ ID NO: 11 - DNA, 65 bp, artificial sequence
  primer
ggggacaagtttgtacaaaaaagcaggctaggaggaattaaccatgtatctgcaagacattgacc

SEQ ID NO: 12 - DNA, 50 bp, artificial sequence
  primer
ggggaccactttgtacaagaaagctgggtcagttctattggatgatttcg

SEQ ID NO: 13 - DNA, 1041 bp, Pseudomonas putida
atgacagacaagagccaacaattcgccagcgacaactattccggtatttgccccgaagcctgggcggcgatggaaaaagccaaccgcggccacgaccgcgcctacggtgacgaccagtggaccgagcgcgcatcggagtacttccgcaaactgttcgaaaccgactgcgaagtgttcttcgccttcaacggcaccgccgccaattccctggccctggcgtcgctgtgccagagctatcacagcgtgatctgctccgaaaccgcccacgtcgaaaccgacgaatgcggtgcgccggagtttttctccaacggctccaagctgctgacagcggccagcgtcaacggcaagctaacgccacagtctattcgtgaagtggcgctcaaacgccaggatatccattaccccaagccgcgcgtggtgaccattacccaggccacggaagtgggcacggtgtaccgccccgacgagctgaaggcgatcagcgccacctgcaaggagctgggcctgaacctgcacatggacggcgcacgctttaccaacgcctgtgctttcctgggctgcagcccggccgaactgacctggaaggccggtgtggatgtgctgtgctttggcggcaccaagaacggcatggcggtgggcgaggcgattctgttcttcaaccgccagttggccgaagacttcgattatcgctgcaagcaagccgggcaactggcgtcgaaaatgcgcttcttgtcggcgccatgggtcgggctgctggaagatggcgcctggttacgccatggcaaccacgccaatcattgcgcgcagctgctggcatcgttggtcagtgacctgccgggggtagagctgatgttcccggtggaggccaacggggtgttcctgcagatgccggagcacgccatcgaggcgctgcggggcaagggctggcgcttctatacctttatcggcagcggtggcgcgcgcttcatgtgctcgtgggataccgaagaagcgcgggtgcgtgagctggccgcggatatccgcacgatcattggtggctga

SEQ ID NO: 14 - protein, 346 aa, Pseudomonas putida
MTDKSQQFASDNYSGICPEAWAAMEKANRGHDRAYGDDQWTERASEYFRKLFETDCEVFFAFNGTAANSLALASLCQSYHSVICSETAHVETDECGAPEFFSNGSKLLTAASVNGKLTPQSIREVALKRQDIHYPKPRVVTITQATEVGTVYRPDELKAISATCKELGLNLHMDGARFTNACAFLGCSPAELTWKAGVDVLCFGGTKNGMAVGEAILFFNRQLAEDFDYRCKQAGQLASKMRFLSAPWVGLLEDGAWLRHGNHANHCAQLLASLVSDLPGVELMFPVEANGVFLQMPEHAIEALRGKGWRFYTFIGSGGARFMCSWDTEEARVRELAADIRTIIGG

SEQ ID NO: 15 - DNA, 39 bp, artificial sequence
  primer
gtgcaccgtctcccatgacagacaagagccaacaattcg

SEQ ID NO: 16 - DNA, 45 bp, artificial sequence
  primer
gtgcaccgtctcctcgagtcagccaccaatgatcgtgcggatatc

SEQ ID NO: 17 - protein, 466 aa, Escherichia coli
MDQKLLTDFRSELLDSRFGAKAISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 18 - protein, 466 aa, Escherichia coli
MDKKQVTDLRSELLDSRFGAKSISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT